(19) United States
(12) Patent Application Publication
(10) Pub. No.: US 2003/0181940 A1
(43) Pub. Date: Sep. 25, 2003

(54) Ventricular restoration shaping apparatus and method of use

(76) Inventors: Gregory Murphy, Annandale, VA (US); Mitta Suresh, Richardson, TX (US); Albert Michael Davis, Richardson, TX (US)

Correspondence Address: ERIC B. MEYERTONS, MEYERTONS, HOOD, KIVLIN,, KOWERT & GOETZEL, P.C., P.O. BOX 398, AUSTIN, TX, 78767-0398, US

(21) Appl. No.: 10/350,297

(22) Filed: Jan. 23, 2003

Related U.S. Application Data
(63) Continuation-in-part of application No. 09/864,510, filed on May 24, 2001.
(60) Provisional application No. 60/272,073, filed on Feb. 28, 2001.
(60) Provisional application No. 60/351,069, filed on Jan. 23, 2002.

(51) Int. Cl. A61M 29/00

(52) U.S. Cl. 606/194

(57) ABSTRACT

A device, or apparatus, is described for use as a guide to reconstruct an enlarged left ventricle of a heart to the shape of an appropriate left ventricle. The device may be a shaper. In one embodiment, a shaper may have a shape substantially different than the shape of an appropriate left ventricle of a heart. The shaper may be positionable in a left ventricle of the heart for use during reconstruction of the left ventricle. In certain embodiments, the shaper may include an upper portion substantially similar in shape to an upper portion of the appropriate left ventricle and an apex portion substantially similar in shape to an apex portion of the appropriate left ventricle. In some embodiments, an attachment may be coupled to the shaper and/or the shaper may include an access port.

### PRIORITY CLAIM

This application is a continuation-in-part of U.S. patent application Ser. No. 09/864,510 entitled “Ventricular Restoration Shaping Apparatus and Method of Use” filed on May 24, 2001, which claims priority to Provisional Patent Application No. 60/272,073 entitled “Ventricular Restoration Shaping Apparatus and Method of Use” filed on Feb. 28, 2001. This application further claims priority to Provisional Patent Application No. 60/351,069 entitled “Ventricular Restoration Shaping Apparatus and Method of Use” filed on Jan. 23, 2002.

### RELATED PATENTS

This patent application incorporates by reference in its entirety U.S. patent application Ser. No. 09/864,510, filed by the common assignee of the application on May 24, 2001.

### BACKGROUND OF THE INVENTION

1. Field of the Invention

This invention relates generally to surgical methods and apparatus for performing surgical ventricular repair on a dilated left ventricle.

2. Description of the Related Art

The function of a heart in an animal is primarily to deliver life-supporting oxygenated blood to tissue throughout the body. This function is generally accomplished in four stages, each relating to a particular chamber of the heart. Initially, deoxygenated blood is received in the right auricle of the heart. This deoxygenated blood is pumped by the right ventricle of the heart to the lungs where the blood is oxygenated. The oxygenated blood is initially received in the left auricle of the heart and ultimately pumped by the left ventricle of the heart throughout the body. The left ventricular chamber of the heart is of particular importance in the blood pumping process as the left ventricular chamber is relied upon to pump the oxygenated blood initially through an aortic valve to and ultimately throughout the entire vascular system of the body.

The shape and volume of a normal heart are of particular interest as these features combine to dramatically affect the way that the blood is pumped. The left ventricle, which is the primary pumping chamber, is typically somewhat elliptical, conical or apical in shape (i.e., it is longer, long axis longest portion from aortic valve to apex, than it is wide, short axis widest portion from ventricle wall to septum, and descends from a base with a decreasing cross-sectional circumference, to a point or apex). The left ventricle is further defined by a lateral ventricle wall and a septum that extends between the auricles and the ventricles. The left ventricle is also composed of the aortic valve, papillary muscles, chordae tendinae, and mitral valve. The function of the left ventricle depends on all of these components being properly aligned to ensure maximum ventricle performance.

Two types of motion accomplish the pumping of the blood from the left ventricle. One of these motions is a simple squeezing motion that occurs between the lateral wall and the septum. The squeezing motion occurs as a result of a thickening of the muscle fibers in the myocardium. This squeezing motion compresses the blood in the ventricle chamber and ejects the blood into the body. The thickening of the muscle fibers may change between diastole and systole. This thickening may be seen easily by echocardiogram, PET, and/or MRI imaging, and the thickening can be routinely measured.

The other type of motion is a twisting or writhing motion that begins at the apex and rises toward the base. The rising writhing motion occurs because the heart muscle fibers run in a circular or spiral direction around the heart. When these fibers constrict, they cause the heart to twist initially at the small area of the apex but progressively and ultimately to the wide area of the base. These squeezing and twisting motions are equally important as they are each responsible for moving approximately one-half of the blood pumped. The contractility or stiffness of these fibers are major determinants in how well the ventricle pumps.

The amount of blood pumped from the left ventricle divided by the amount of blood available to be pumped is generally referred to as the ejection fraction of the heart. Typically, a healthier heart has a higher ejection fraction. A normal heart, for example, may have a total volume of one hundred milliliters and an ejection fraction of sixty percent. Under these circumstances, 60 milliliters of blood are pumped with each beat of the heart. It is this volume in the normal heart of this example that is pumped with each beat to provide nutrients including oxygen to the muscles and other tissues of the body. Realizing that the heart is part of the body tissue and that heart muscle also requires oxygenated blood, it can be appreciated that the normal function of the heart is greatly upset by clotting or closure of the coronary arteries. When the coronary arteries are blocked, an associate portion of the heart muscle becomes oxygen-starved and begins to die. This process is clinically referred to as a heart attack. Ischemic cardiomyopathy (ischemia) typically occurs as the rest of the heart dilates in an attempt to maintain the heart's output to the body.

As the ischemia progresses through its various stages, the affected myocardium dies, thus losing its ability to contribute to the pumping action of the heart. The ischemic muscle is no longer capable of contracting, and thus cannot contribute to either the squeezing or twisting motion required to pump blood. This non-contracting tissue is said to be akinetic. In severe cases, the akinetic tissue, which is not capable of contracting, is in fact elastic so that blood pressure tends to develop a bulge or expansion of the chamber. This muscle tissue is not only akinetic, in that it does not contribute to the pumping function, but it is in fact dyskinetic, in that it detracts from the pumping function. This is particularly detrimental as the limited pumping action available causes the heart to lose even more of its energy by pumping the bulge instead of the blood.

The body seems to realize that with a reduced pumping capacity, the ejection fraction of the heart is automatically reduced. For example, the ejection fraction may drop from a normal sixty percent to perhaps twenty percent. Realizing that the body still requires the same volume of blood for oxygen and nutrition, the body forces the heart to dilate or enlarge in size so that the smaller ejection fraction pumps about the same amount of blood. As noted, a normal heart with a blood capacity of seventy milliliters and an ejection fraction of sixty percent would pump approximately 42 milliliters per beat. The body seems to appreciate that this same volume per beat can be maintained by an ejection fraction of only thirty-percent if the ventricle enlarges to a capacity of 140 milliliters. This increase in volume, commonly referred to as “remodeling”, not only changes the volume of the left ventricle, but also its shape. The heart becomes greatly enlarged and the left ventricle becomes more spherical in shape and loses its apex.

On the level of the muscle fibers, it has been noted that dilation of the heart causes the fibers to reorient themselves so that they are directed away from the inner heart chamber containing the blood. As a consequence, the fibers are poorly oriented to accomplish even the squeezing action, as the lines of force become less perpendicular to the heart wall. This change in fiber orientation occurs as the heart dilates and moves from its normal elliptical shape to its dilated spherical shape. The spherical shape further reduces pumping efficiency since the fibers, which normally encircle the apex and facilitate writhing, are changed to a more flattened formation as a result of these spherical configurations.

Of course, this change in architecture has a dramatic effect on wall thickness, radius, and stress on the heart wall. In particular, absent the normal conical shape, the twisting motion at the apex, which can account for as much as one half of the pumping action, is lost. As a consequence, the more spherical architecture must rely almost totally on the lateral squeezing action to pump blood. This lateral squeezing action is inefficient and very different from the more efficient twisting action of the heart.

Although the dilated heart may be capable of sustaining life, the heart is significantly stressed and rapidly approaches a stage where it can no longer pump blood effectively. In this stage, commonly referred to as congestive heart failure, the heart becomes distended and is generally incapable of pumping blood returning from the lungs. This complication further results in lung congestion and fatigue. Congestive heart failure is a major cause of death and disability in the United States with approximately 400,000 cases occurring annually.

Following coronary occlusion, successful acute reperfusion by thrombolysis (clot dissolution), percutaneous angioplasty, or urgent surgery can decrease early mortality by reducing arrhythmias and cardiogenic shock. Addressing ischemic cardiomyopathy in the acute phase (e.g., with reperfusion) may salvage the epicardial surface. Although the myocardium may be rendered akinetic, at least the myocardium may not be dyskinetic. Post-infarction surgical re-vascularization can be directed at remote viable muscle to reduce ischemia. However, post-infarction surgical re-vascularization does not address the anatomical consequences of the akinetic region of the heart that is scarred. Despite these techniques for monitoring ischemia, cardiac dilation and subsequent heart failure continue to occur in approximately fifty percent of post-infraction patients discharged from the hospital.

Various surgical approaches have been taken to primarily reduce the ventricular volume. Some of these procedures involve removing dyskinetic and akinetic regions of the heart, then surgically joining the viable portions of the myocardial walls, typically with the use of a patch surgically placed in the walls using a Fontan stitch.

These surgical procedures have been met with some success as the ejection fraction has been increased, for example, from twenty-four percent to forty-two percent. These procedures also have shown that even the most experienced surgeons can incorrectly repair a dilated ventricle. Recent studies have shown that the most experienced surgeon in this procedure has induced mitral regurgitation in 33% of one patient group that didn't have mitral regurgitation prior to surgery, due to their ventricles being too spherical after surgery. The studies also show significant variability among patients, as some patients improve and others do not improve. Some patients whose ventricles are made too small are even worse off after surgery than before surgery. The difficulty of reconstructing a dilated ventricle has kept the procedure restricted to a very small group of surgeons who are very experienced in the procedure.

Another problem with the current procedure for repairing dilated ventricles is that the procedure does not take into account the fact that the left ventricle is composed of many different components. The current procedure also looks at the ventricle as it exists before surgery and treats that ventricle without considering what the effects of surgery will be on the other parts of the ventricle. These two problems may lead to the surgeon reducing the volume of the ventricle by making the ventricle more spherical since the surgeon may not take into consideration shape. These problems can also induce mitral regurgitation in 33% of patients since the surgeon doesn't take into consideration the effect the surgery will have on the mitral valve apparatus.

The aortic valve orientation to the left ventricle changes as people age. The aortic valve orientation goes from being an almost 180 degree angle from the septal wall to the center of the aortic valve in younger patients to approaching 90 degrees for the same angle in elderly patients. The ventricle is designed to push blood out through the aortic valve. If the ventricle is not aligned with the aortic valve, turbulence will be created in the ventricle, thus reducing the blood flow and forcing the ventricle to work harder. The current approach for reconstructing a dilated ventricle does not consider the position and angle of the aortic valve.

What is needed therefore is a reliable method and apparatus to allow a surgeon to perform surgical ventricular restoration without having to guess at the proper size, shape, and orientation of the ventricle components. In response to these and other problems, an improved apparatus and method is provided for surgical ventricular repair.

### SUMMARY

In an embodiment, a shaping device (e.g., a shaping mannequin) may be used by a surgeon to reshape a left ventricle to the shape and size of an appropriate left ventricle. The shaping device may have an anchoring means to hold the shaping device in appropriate geometric relationship with other ventricular apparatus. The ventricular apparatus may include an aortic valve, a mitral valve, papillary muscles, and/or chordae tendinae. In one embodiment, the anchoring means is an appendage to the shaping device that snugly fits into aortic or mitrial valve. The anchoring means may also include two or more appendages to the shaping device. For example, one appendage may snugly fit into the mitrial valve while a second appendage fits into the aortic valve. The shaping device may be a preshaped balloon that can be deflated and inflated to the required shape and size. In some embodiments, the shaping device may be a wire frame. The shaping device may also include a length measuring means. Marking on the length measuring means may be equidistant markings.

The procedure for using the shaping device addresses the ability of the surgeon to perform a surgical ventricular repair procedure. The shaping device procedure allows the surgeon to ensure that the intended size and shape of the ventricle are achieved. The shaping device procedure may also allow the surgeon to achieve the intended orientation and size of the aortic and mitral valves as well as the chordae tendinae and papillary muscles.

The shaping device procedure may greatly increase the efficiency of the heart and significantly reduce stress on the heart muscle and improve surgical outcome by allowing the surgeon to correct all the components of the ventricle to the intended proportions. The procedure also allows the surgeon to make the procedure repeatable and reliable by involving a precise device (i.e., the shaping device) and taking variation out of the surgical procedure.

The shaping device may include one or more components. One component may be a shaping mandrel of a known volume in the shape intended for the ventricle. Other components may include protuberances or attachments that fit onto the shaping device and fit into either the mitral or aortic valve, or fit into both valves. Another component may be a measuring device that allows the surgeon to assess the angle of the papillary muscles and chordae tendinae to the mitral valve. A measuring device may also allow the surgeon to check the length of the chordae tendinae and the papillary muscles. In certain embodiments, one measuring device may be used for both assessing the angle of the papillary muscles and chordae tendinae to the mitral valve and checking the length of the chordae tendinae and the papillary muscles. In some embodiments, the shaping mandrel may be clear and have graduated scales printed on it to allow the surgeon to measure and, if necessary, correct the papillary muscles and chordae tendinae.

### BRIEF DESCRIPTION OF THE DRAWINGS

Advantages of the present invention may become apparent to those skilled in the art with the benefit of the following detailed description of the preferred embodiments and upon reference to the accompanying drawings in which:

FIG. 1 depicts a side view of an embodiment of a shaping device.

FIG. 2 depicts a side view of a balloon embodiment of a shaping device.

FIG. 3 depicts a section view of another balloon embodiment of a shaping device.

FIG. 4 depicts a side view of a wire frame embodiment of a shaping device in an expanded condition.

FIG. 5 depicts a side view of a wire frame embodiment of a shaping device in a collapsed condition.

FIG. 6 depicts a section view cut transversely through the embodiment of FIG. 5.

FIG. 7 depicts a front view of another embodiment of a shaping device.

FIG. 8 depicts a section view of the embodiment illustrated in FIG. 7.

FIG. 9 depicts another embodiment of a shaping device.

FIG. 10 depicts another embodiment of a shaping device.

FIG. 11 depicts an embodiment of a shaping device with two protuberances or attachments for each valve.

FIG. 12 depicts an embodiment of a shaping device with two protuberances for the aortic valve with measurement devices for the papillary muscles and chordae tendinae.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

A Basic Shaping Device:

The shape of a normal heart is of particular interest as the shape dramatically affects the way that the blood is pumped. The left ventricle, which is the primary pumping chamber, is somewhat conical or apical in shape. The left ventricle is longer (long axis longest portion from aortic valve to apex) than it is wide (short axis widest portion from ventricle wall to septum) and descends from a base with a decreasing cross-sectional circumference to a point or apex. The left ventricle is further defined by a lateral and posterior ventricle wall and a septum that extends between the auricles and the ventricles.

Pumping of blood from the left ventricle is accomplished by two types of motion. One of these motions is a simple squeezing motion that occurs between the lateral wall and the septum. The squeezing motion occurs as a result of a thickening of the muscle fibers in the myocardium. The squeezing motion compresses blood in the ventricle chamber and ejects blood into the body. The thickness of the muscle fibers changes as the ventricle contracts. The change in thickness may easily be seen and/or routinely measured by echocardiogram and other techniques.

The other type of ventricular motion is a twisting or writhing motion. The twisting or writhing motion may begin at the apex and rise toward the base of the ventricle. The rising writhing motion occurs because the heart muscle fibers run in a circular or spiral direction around the heart. When these fibers constrict, they cause the heart to twist initially at the small area of the apex. The twisting, however, progressively and ultimately moves to the wide area of the base. The squeezing and twisting motions are equally important, as they are each responsible for moving approximately one-half of the blood pumped.

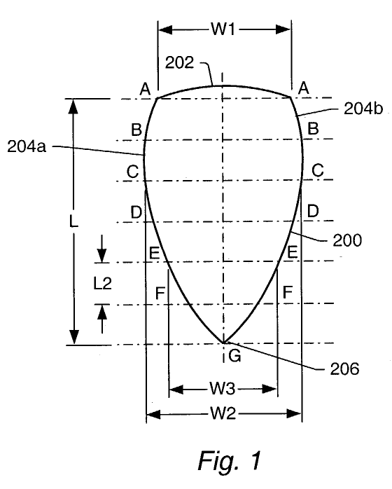

Turning now to FIG. 1, shaping device 200 may be used to allow the left ventricle to be reconstructed back to a pre-enlarged operating condition. When a surgeon uses shaping device 200 as a guide in reconstructing the left ventricle, the reconstructed heart may be formed closer to the size and shape of the pre-enlarged heart. Consequently, the heart may perform better post-operatively than with typical conventional enlarging methods. As shown in FIG. 1, shaping device 200 may generally be conical or “tear drop” in shape. The length “L” of shaping device 200 may vary with each patient and, typically, is a function of the volume deemed appropriate for the patient after the ventricle has been restored. Depending on the patient, the length “L” may be range from about 3 inches to about 4 inches to generally match the length of the pre-enlarged left ventricle. A surgeon may select the appropriate volume for the shaping device by estimating the volume of the pre-enlarged left ventricle. The appropriate volume of the pre-enlarged left ventricle for a patient may be estimated. Generally, the volume of the pre-enlarged left ventricle is estimated to be about 50 cc per square meter to about 70 cc per square meter of body surface area. The body surface area may be estimated according to the following formula, as is generally known in the art:

BSA=0.001*71.84w⁰⁴²⁸*h⁰⁷²⁵ (1)

Where: BSA=body surface area;

w=body weight in kilograms; and

h=body height in centimeters.

1The shaping device may be of a shape such that when the surgeon inserts the shaping device into the left ventricle, the surgeon can use the shaping device to remodel the left ventricle into the “appropriate shape and volume” for a patient. In other words, the shaping device may be of a shape similar to the shape of the left ventricle cavity. As described herein, other aspects of a shaping device may allow a surgeon to model the left ventricle without the shaping device actually being the shape of the left ventricle. In one embodiment, shaping device 200 may be a generally conical shaped object composed of portions of spherical elements having different radii. Other embodiments may include shapes such as, but not limited to, “pear” shape, elliptical, and “tear drop” shape.

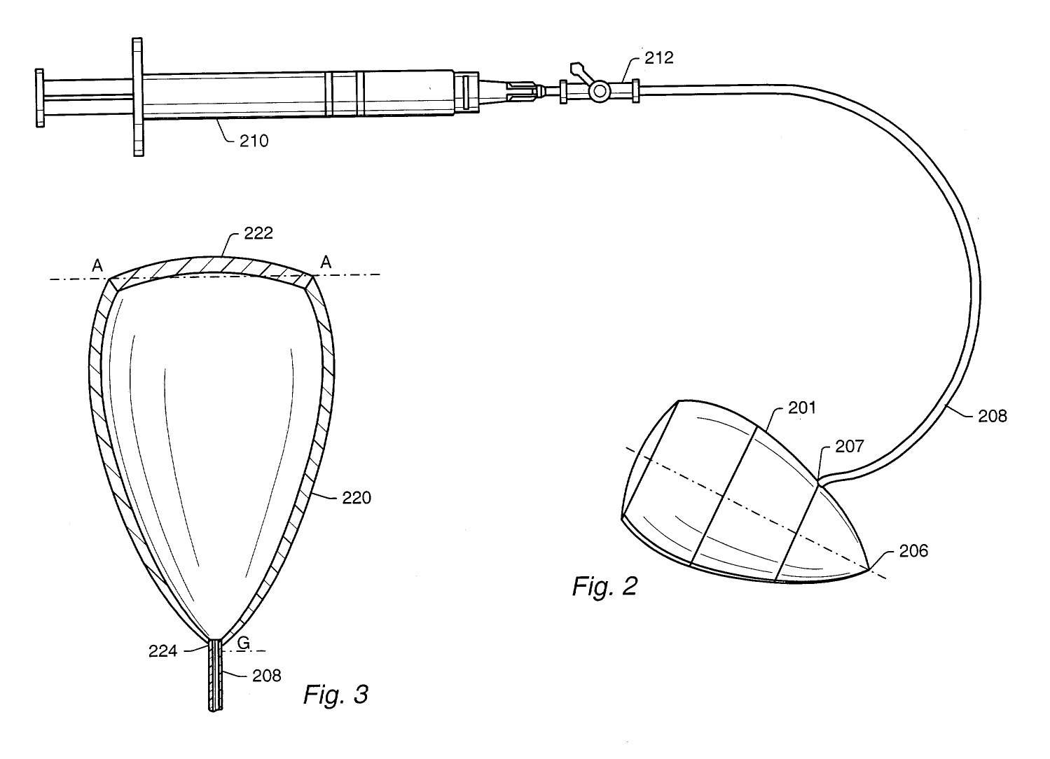

In some embodiments, such as illustrated in FIG. 2, the shaping device may be inflatable balloon 201. Balloon 201 may have a thickness in the range of about 0.002 inches to about 0.08 inches (e.g., about 0.03 inches). Balloon 201 may have a thickness of less than about 0.08 inches. A distal end of filler tube 208 may be coupled to point 207 along the exterior surface of balloon 201. For instance, point 207 may be located approximately ⅓ along a length of balloon 201, as illustrated in FIG. 2. In other embodiments, filler tube 208 may be coupled to balloon 201 at vertex 206 or another suitable location along a length of balloon 201. Filler tube 208 may be made of materials commonly used in the art (e.g., PVC or other biocompatible materials).

A proximal end of filler tube 208 may be connected to fluid reservoir 210. In an embodiment, fluid reservoir 210 is a syringe. Fluid reservoir may be used to provide a pre-specified amount of fluid to balloon 201 through filler tube 208. For example, a syringe may inject a pre-specified amount of fluid into balloon 201. In some embodiments, a fluid control device 212 (e.g., a stopcock) may be coupled to the distal end of filler tube 208. The injection of fluid through filler tube 208 may inflate balloon 201 to an inflated condition, as illustrated in FIG. 2. Once inflated, the fluid inside the shaping device may be inhibited from escaping by locking fluid control device 212. Locking fluid control device 212 may allow balloon 201 to stay inflated with the proper volume, shape, and contour during a reconstruction procedure.

The fluid pressure inside balloon 201 may be monitored by a pressure transducer (e.g., a piezoelectric transducer). The pressure transducer may be coupled to filler tube 208 with a y-connection. One lead of the y-connection may be coupled to a pressure monitor and the other lead may be coupled to the fluid source. Alternatively, the pressure monitor may be coupled to a three-way stopcock that would monitor the pressure on the filler tube side of the three-way stopcock.

The fluid used to fill balloon 201 may be any one of a number of fluids such as, but not limited to, saline solution or distilled water. Some embodiments may use a sealed balloon containing a silicone gel, such as a liquid methyl silicone resin, capable of being vulcanized blended with a dimethyl silicone fluid. Such gels are available from Applied Silicon, Inc. (Ventura, California). An embodiment using a sealed balloon may not need an external fluid reservoir such as fluid reservoir 210.

Balloon 201 may be conventionally formed on a mandrel. The mandrel may have dimensions corresponding to the shape, contour, and size of the shaping device. As is known in the art, the mandrel can be made of metal, glass, or a hardened gelatin. To form balloon 201, the mandrel may be dipped into a polymer solution that leaves a thin polymer coating on the mandrel surface. After the polymer has cured, balloon 201 may be removed by peeling the thin coating off the mandrel or by flushing mandrel material out of the shaping device.

Shaping Device—Other Embodiments:

The shaping device may be made out of a variety of materials in a number of configurations creating a number of embodiments. In an embodiment, if the shaping device is molded from a thermoplastic polymer such as PVC, polyethylene, or a similar material, the balloon may be “non-expandable” when inflated. Once the thermoplastic polymer balloon is inflated, balloon 201 will not significantly expand beyond the original shape. To illustrate, several shaping devices might have volumes ranging from about 100 cc to about 150 cc at 10 cc increments. If a surgeon predetermines that a patient's pre-enlarged left ventricle was 128 cc, then the surgeon might select a non-expandable balloon having a volume of 130 cc. A surgeon may also request a custom non-expandable balloon with a volume specifically sized for an individual patient.

In other embodiments, if balloon 201 is made from an elastomeric material, the balloon may significantly expand. Such elastomeric materials may include latex, polyurethane, silicone, and other elastomers sold under the trade names KRATON™ (Shell Chemical, New York, N.Y. or KRATON Polymers, Houston, Tex.), C-FLEX® (Concept Polymer, Largo, Fla.) and SANTOPRENE® (Monsanto, St. Louis, Mo.). Once the balloon is substantially inflated, the influx of additional fluid causes additional expansion of the balloon. In this embodiment, a surgeon may simply inflate the balloon to a specific volume. The original shape of the balloon may be maintained during this expansion by selectively thickening the walls of the balloon.

FIG. 3 depicts a section view of an embodiment showing thickened walls of “expandable” balloon 220. An insertion or distal end 222 of balloon 220 may have walls at a maximum thickness. From the line A-A, the wall thickness progressively decreases to vertex 224 at point G. In some embodiments, vertex 224 connects to filler tube 208. The wall thickness may depend on the expansion range of the balloon. For example, for an expansion of about 100 cc to about 150 cc, the thickness of the balloon would vary from about 0.01 inches at a thin end to about 0.05 inches at a thick end. Thus, the size or volume of balloon 220 may be controlled by controlling the amount and pressure of the fluid injected into the balloon.

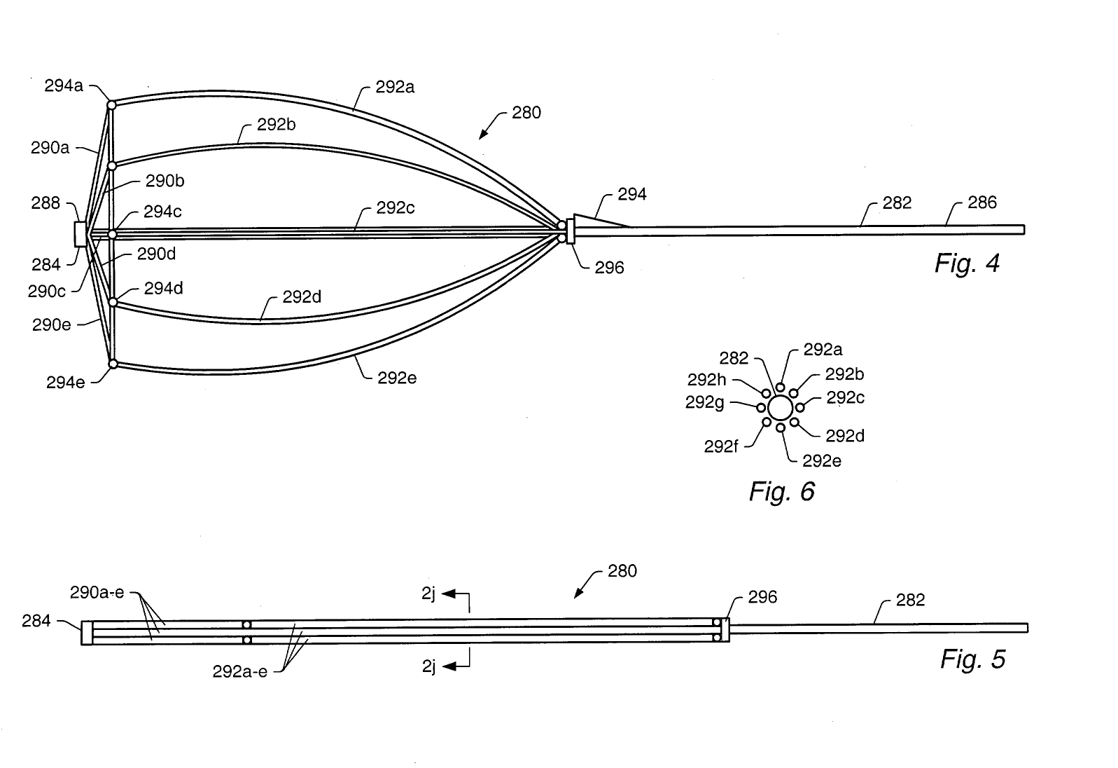

A shaping device may not be limited to polymeric balloon embodiments. FIG. 4 depicts shaping device 280 made from a wire skeleton or frame. The wire frame may be made from surgical grade stainless steel, titanium, tantalum, and/or nitinol, which is a commercially available nickel-titanium alloy material that has shape memory and is superelastic. Nitinol medical products are available from AMF of Reuilly, France, and Flexmedics Corp., of White Bear Lake, Minn.

Shaping device 280, depicted in FIG. 4, is in an expanded condition. Running through the center of shaping device 280 is main shaft 282. Main shaft 282 may have distal end 284 and proximal end 286. At distal end 284 may be joint 288. Coupled to joint 288 may be a series of back ribs 290a though 290h (only back ribs 290a through 290e are visible in FIG. 4). Back ribs 290a through 290h may be connected to front ribs 292a-292h by hinges 294a though 294h (only front ribs 292a-292e and hinges 294a-294e are visible in FIG. 4). The proximal end of front ribs 292a through 292e may be connected to collar 296 through a series of hinges radially spaced around the collar. The use of hinges around collar 296 may encourage front ribs 292a-292h to form a convex angle with respect to shaft 282 at the collar.

FIG. 5 depicts shaping device 280 in a collapsed position. In a collapsed position, back ribs 290a-290h and front ribs 292a-292h surround shaft 282, as illustrated in FIG. 5. FIG. 6 depicts a section view cut transversely through shaft 282 and the front ribs 292a-292h. In operation, once shaping device 280 is inserted into the left ventricle, a surgeon may slide collar 296 along shaft 282 towards distal end 284. The force exerted on collar 296 may cause the ribs to buckle radially outward, as illustrated in FIG. 4. Eventually, the front ribs 292a-292h will bend under the applied force. Because the front ribs 292a-292h are under stress, they may tend to push collar 296 towards proximal end 286. Lock 294 may inhibit any movement towards proximal end 286. Thus, collar 296 may be held firmly in place along shaft 282 by front ribs 292a-292h exerting a force through collar 296 to lock 294. Lock 294 may be spring activated and designed such that collar 296 may easily slide over the lock when moving from proximal end 286 to distal end 288. When a surgeon is ready to remove shaping device 280, the surgeon may collapse the shaping device by pressing down on lock 294, which allows collar 296 to slide past the lock towards proximal end 286.

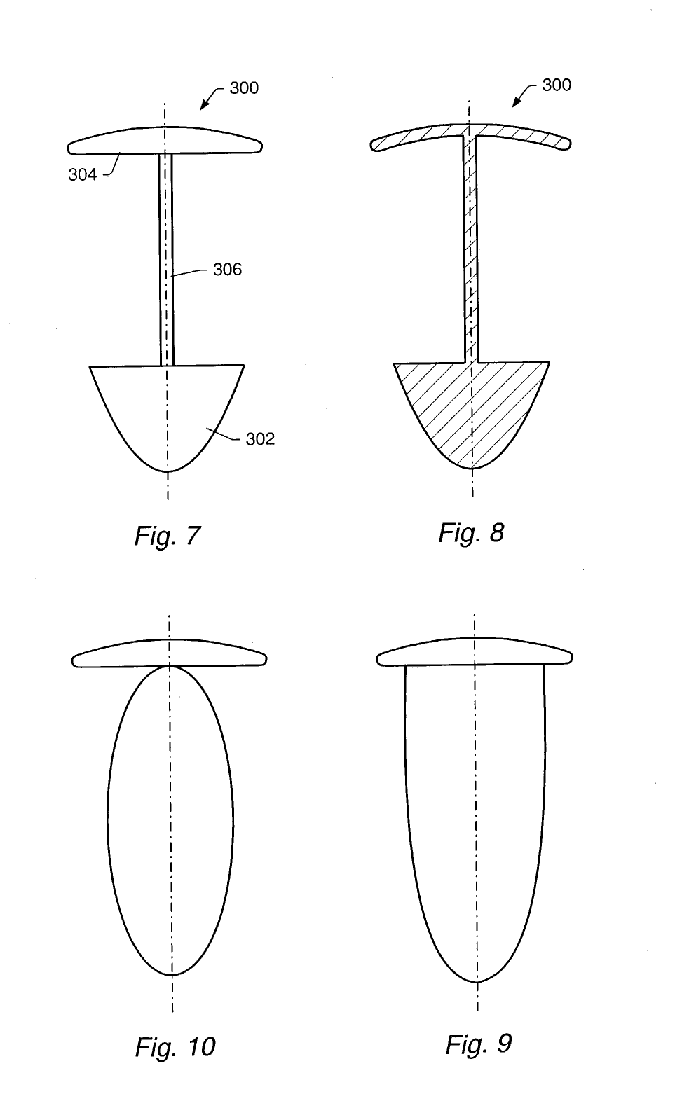

In certain embodiments, the shaping device may be used as a general guide for a surgeon. Thus, the shaping device may not need to have the same shape and volume as the appropriate left ventricle. The shaping device may have a differing shape that is used as a guide so that a surgeon can reconstruct the patients ventricle to the size and shape of the appropriate left ventricle. FIG. 7 depicts such an embodiment. In the embodiment of FIG. 7, shaping device 300 has cone shaped member 302 and dome shaped member 304. Cone shaped member 302 may be connected to dome-shaped member 304 by central mandrel 306. FIG. 8 depicts a cross-sectional view of shaping device 300 depicted in FIG. 7. Cone shaped member 302 may be approximately the same size and shape as the apex portion of shaping device 200, depicted in FIG. 1. Additionally, dome-shaped member 304 is approximately the same size and shape as the upper portion of shaping device 200. Thus, cone shaped member 302 and dome-shaped member 304 may have substantially similar sizes and shapes as the corresponding apex portions and upper portions of the left ventricle of the heart. The overall length of shaping device 300 may be about the same length as shaping device 200. Thus, when inserted into the left ventricle during a surgical procedure, shaping device 300 may be used as a guide so that the surgeon can reconstruct the patient's left ventricle to be the same size and shape as the appropriate left ventricle. Shaping device 300 may be made of a semi-rigid material (e.g., plastic). The shaping device 300 may also be made from a flexible material such as, but not limited to, soft plastic, silicone, or rubber.

FIG. 9 depicts an embodiment of shaping device 400, which may be used as a guide to shape a patient's left ventricle. In FIG. 9, shaping device 400 may be an expandable device, such as shaping device 200, or a solid device, such as shaping device 300. A variety of shape possibilities exist for a shaping device. An expandable device may expand to a predetermined shape that may be substantially similar in shape to an appropriate left ventricle or may be a different shape than the appropriate left ventricle. A shaping device, either expandable or solid, of substantially similar shape and size as the appropriate left ventricle may be used as a pattern for reconstruction of the left ventricle. In some embodiments, a shaping device of a different shape from the appropriate left ventricle may be used as either a pattern or a guide for reconstructing the left ventricle. One example of another embodiment of a shape for a shaping device is depicted in FIG. 10. Shaping devices of various shapes and sizes may have varying wall thickness, cross-sections, and/or other dimensions as described herein.

A Shaping Device with Valve Protrusions:

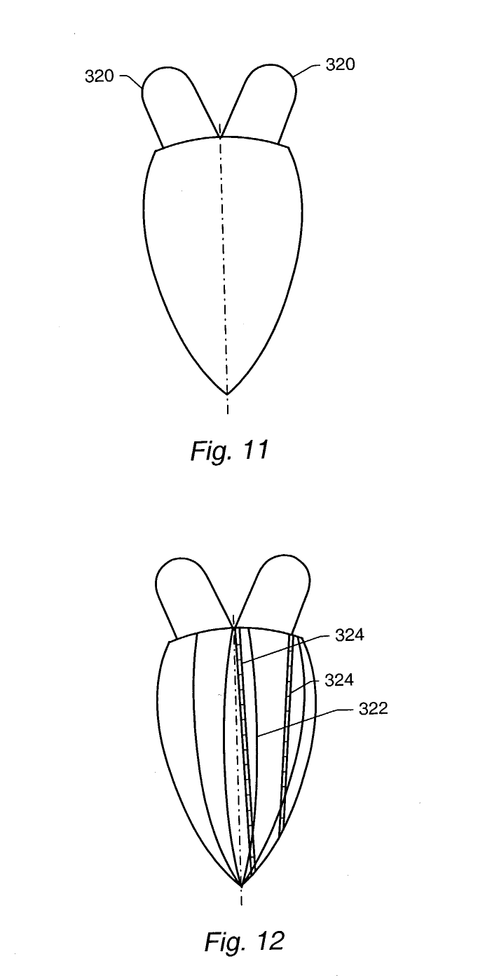

Certain embodiments may also include a mechanism that holds on to attachments that fit into valve openings of the patient's heart. In one embodiment, a shaping device may have a means for fitting attachments 320 of different sizes and angles to the shaping device, as depicted in FIG. 11. Such a mechanism may allow a surgeon the flexibility to customize an intended shape for a particular patient. One patient may have a ventricle that the surgeon desires to reconstruct where a volume of about 120 cc is desired, with an angle from the ventricle to the aortic valve of about 130° and a mitral valve that is about 24 cm diameter. The surgeon could then select a 120 cc shaping device and combine it with an attachment that matches the intended ventricle to aortic valve angle and combines the attachment with a 24 cm attachment to fit the mitral valve. This shaping device may also have graduated markings for measuring and correcting the papillary muscles and chordae tendinae angles and lengths.

In some embodiments, the shaping device may have grooves on one side to accommodate the papillary muscles and the chordae tendinae. This shaping device may also have the valve protrusions integrally included or included as attachments.

One embodiment may have a shaping device with access port 322, depicted in FIG. 12, to allow the surgeon to access the papillary muscles and chordae tendinae with the shaping device in place. The surgeon may examine the interior of the ventricle through the access port (i.e., perform an endoscopy on the ventricle). In some embodiments, the surgeon may use a fiber optic device to perform the endoscopy. In certain embodiments, the ventricle shaping device may be in the form of a wire frame with two protuberances and with measurement markers 324 for papillary muscles and chordae tendinae, as depicted in FIG. 12.

During operation, a surgeon may determine the size, shape, and orientation intended for reconstructing the ventricle. During the surgical procedure, the surgeon may then open the ventricle and note the extent of a scar inside the ventricle. The desired volume-shaping device may then be placed in the ventricle and the attachments placed in either both the valves, or just one valve. The surgeon may then secure the ventricle around the shaping device. The surgeon may assess the orientation of the chordae tendinae and papillary muscles to the aortic valve using the graduated measuring devices on the mandrel. The surgeon may also use a separate measuring device on the mandrel to measure the length of the papillary muscles and chordae tendinae. The surgeon may adjust the components as needed and measure the new alignment and length after the repair.

In certain embodiments, cardiac image processing computer programs may be used in combination with specific information about a patient (e.g., specific pre-operation disease state information such as, but not limited to, ventricle size, shape, and orientation data, which may be obtained through a radiological exam) to pre-operatively determine the shape and size of a shaping device specifically for that patient. Image processing computer programs may be specifically formulated for imaging the heart (e.g., programs such as those available from Chase Medical (Richardson, Tex.)). A radiological exam may employ various diagnostic imaging modalities such as, but not limited to, ultrasound (US), computed tomography (CT), magnetic resonance imaging (MRI), and nuclear medicine (Nmed). Pre-operatively determining the shape and size of a shaping device based on a particular patient's criteria may allow a manufacturer to customize the shaping device for each individual patient. In some embodiments, information obtained about the shape and size of the shaping device for an individual patient may be transferred (e.g., communicated electronically) to a manufacturing center that can use the information to design and manufacture the specific shaping device for the patient. The manufacturing center may use the information in an automated manufacturing system for producing the shaping device. An automated manufacturing system may include computer programs that control and/or provide input to the manufacturing process. The produced, patient specific, shaping device may later be used in a surgical reconstruction of a ventricle for the individual patient.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. A shaping system, comprising:
a shaper having a shape substantially different than the shape of an appropriate left ventricle of a heart, wherein the shaper is positionable in a left ventricle of the heart during use, wherein the shaper comprises an upper portion substantially similar in shape to an upper portion of the appropriate left ventricle, and wherein the shaper comprises an apex portion substantially similar in shape to an apex portion of the appropriate left ventricle.

2. The shaping system of claim 1, wherein the shaper has a substantially similar length as the appropriate left ventricle.

3. The shaping system of claim 1, wherein the shaper has a substantially similar width as the appropriate left ventricle.

4. The shaping system of claim 1, wherein the shaper has a substantially similar size as the appropriate left ventricle.

5. The shaping system of claim 1, wherein the upper portion of the shaper comprises a dome shape.

6. The shaping system of claim 1, wherein the apex portion of the shaper comprises a cone shape.

7. The shaping system of claim 1, wherein the upper portion of the shaper and the apex portion of the shaper are coupled by a central portion.

8. The shaping system of claim 7, wherein the central portion comprises a mandrel.

9. The shaping system of claim 7, wherein the central portion comprises an elongated portion.

10. The shaping system of claim 7, wherein the central portion comprises a diameter substantially smaller than a diameter of the appropriate left ventricle.

11. The shaping system of claim 1, wherein the shaper comprises an overall length substantially similar to an overall length of the appropriate left ventricle.

12. The shaping system of claim 1, wherein the shaper is used as a guide to reconstruct a left ventricle to the shape and size of the appropriate left ventricle during use.

13. The shaping system of claim 1, wherein the shaper has a short axis and a long axis.

14. The shaping system of claim 1, further comprising an attachment configured to be coupled to the shaper.

15. The shaping system of claim 14, wherein the attachment is coupled to the shaper.

16. The shaping system of claim 14, wherein the attachment is configured to fit into a heart valve.

17. The shaping system of claim 14, wherein the attachment is configured to be coupled to the shaper at a selected angle.

18. The shaping system of claim 14, wherein the attachment is configured to be coupled to the shaper at a selected angle, and wherein the selected angle comprises an angle from the left ventricle to a heart valve.

19. The shaping system of claim 14, wherein the attachment comprises a selected length.

20. The shaping system of claim 14, wherein the attachment comprises a selected diameter.

21. The shaping system of claim 14, wherein the attachment is integrally included in the shaper.

22. The shaping system of claim 1, wherein the shaper comprises markings.

23. The shaping system of claim 1, wherein the shaper comprises graduated markings for measuring and correcting papillary muscles and chordae tendinae angles and lengths during use.

24. The shaping system of claim 1, wherein the shaper comprises grooves to accommodate papillary muscles and chordae tendinae.

25. The shaping system of claim 1, wherein the shaper comprises an access port.

26. The shaping system of claim 25, wherein the access port allows a surgeon to access papillary muscles and chordae tendinae during use.

27. The shaping system of claim 25, wherein the access port allows a surgeon to perform an endoscopy during use.

28. The shaping system of claim 1, wherein the shaper comprises flexible material.

29. The shaping system of claim 1, wherein the shaper comprises plastic.

30. A shaping system, comprising:
a shaper having a shape different than the shape of an appropriate left ventricle of a heart, wherein the shaper is positionable in a left ventricle of the heart during use, and wherein the shape of the shaper is used as a guide to allow a surgeon to reconstruct the left ventricle to the shape of the appropriate left ventricle during use.

31. The shaping system of claim 30, wherein the shaper is configurable to expand to a predetermined shape in the left ventricle during use.

32. The shaping system of claim 30, wherein the shaper comprises an expandable balloon.

33. The shaping system of claim 30, wherein the shaper comprises an expandable balloon, and wherein the expandable balloon has a wall thickness of about 0.002 inches to about 0.08 inches.

34. The shaping system of claim 30, wherein the shaper comprises an expandable balloon, and wherein the expandable balloon has a wall thickness of less than about 0.08 inches.

35. The shaping system of claim 30, wherein the shaper comprises an expandable balloon, wherein the expandable balloon has a wall thickness, and wherein the wall thickness selectively varies as a function of the expansion of the balloon.

36. The shaping system of claim 30, wherein the shaper comprises a predetermined contour.

37. The shaping system of claim 30, wherein the shaper is configured to contain at least one fluid in at least a portion of the shaper.

38. The shaping system of claim 30, wherein the shaper is configured to contain at least one fluid in at least a portion of the shaper, and wherein the fluid is configurable to expand the shaper to the predetermined shape.

39. The shaping system of claim 30, wherein the shaper is configured to contain at least one fluid in at least a portion of the shaper, wherein the fluid is configurable to expand the shaper to the predetermined shape, and wherein the fluid is a gel.

40. The shaping system of claim 30, wherein the shaper is configured to contain at least one fluid in at least a portion of the shaper, wherein the fluid is configurable to expand the shaper to the predetermined shape, and wherein the fluid comprises silicone.

41. The shaping system of claim 30, wherein the shaper is configured to inhibit distortion of the predetermined shape when expanded.

42. The shaping system of claim 30, further comprising a tube coupled to the shaper, wherein the tube is configurable to convey a fluid to the shaper.

43. The shaping system of claim 30, further comprising a tube coupled to the shaper, wherein the tube is configurable to convey a fluid to the shaper from a pressurized fluid reservoir.

44. The shaping system of claim 30, further comprising a tube coupled to the shaper, wherein the tube is configurable to convey a fluid to the shaper from a pressurized fluid reservoir, and further comprising a valve coupled to the tube, wherein the valve is configurable to maintain a pressure of the fluid.

45. The shaping system of claim 30, wherein the shaper has a short axis and a long axis.

46. The shaping system of claim 30, wherein the shaper is expanded to the predetermined shape.

47. The shaping system of claim 30, wherein the shaper comprises an upper portion substantially similar in shape to an upper portion of the appropriate left ventricle, and wherein the shaper comprises an apex portion substantially similar in shape to an apex portion of the appropriate left ventricle.

48. The shaping system of claim 30, wherein the shaper has a substantially similar length as the appropriate left ventricle.

49. The shaping system of claim 30, wherein the shaper has a substantially similar width as the appropriate left ventricle.

50. The shaping system of claim 30, wherein the shaper has a substantially similar size as the appropriate left ventricle.

51. The shaping system of claim 30, wherein the shaper comprises an overall length substantially similar to an overall length of the appropriate left ventricle.

52. A shaping system, comprising:
a shaper having a shape different than the shape of an appropriate left ventricle of a heart, wherein the shaper is positionable in a left ventricle of the heart during use; and
an attachment configured to be coupled to the shaper.

53. The shaping system of claim 52, wherein the shape of the shaper is used as a guide to allow a surgeon to reconstruct the left ventricle to the shape of the appropriate left ventricle during use.

54. The shaping system of claim 52, wherein the attachment is coupled to the shaper.

55. The shaping system of claim 52, wherein the attachment is configured to fit into a heart valve.

56. The shaping system of claim 52, wherein the attachment is configured to be coupled to the shaper at a selected angle.

57. The shaping system of claim 52, wherein the attachment is configured to be coupled to the shaper at a selected angle, and wherein the selected angle comprises an angle from the left ventricle to a heart valve.

58. The shaping system of claim 52, wherein the attachment comprises a selected length.

59. The shaping system of claim 52, wherein the attachment comprises a selected diameter.

60. The shaping system of claim 52, wherein the attachment is integrally included in the shaper.

61. The shaping system of claim 52, wherein the shaper has a substantially similar length as the appropriate left ventricle.

62. The shaping system of claim 52, wherein the shaper has a substantially similar width as the appropriate left ventricle.

63. The shaping system of claim 52, wherein the shaper has a substantially similar size as the appropriate left ventricle.

64. The shaping system of claim 52, wherein the shaper comprises an overall length substantially similar to an overall length of the appropriate left ventricle.

65. A shaping system, comprising:
a shaper having a shape different than the shape of an appropriate left ventricle of a heart, wherein the shaper is positionable in a left ventricle of the heart during use, and wherein the shaper comprises an access port.

66. The shaping system of claim 65, wherein the access port allows a surgeon to access papillary muscles and chordae tendinae during use.

67. The shaping system of claim 65, wherein the access port allows a surgeon to perform an endoscopy during use.

68. The shaping system of claim 65, further comprising an attachment configured to be coupled to the shaper.

69. The shaping system of claim 68, wherein the attachment is coupled to the shaper.

70. The shaping system of claim 68, wherein the attachment is configured to fit into a heart valve.

71. The shaping system of claim 68, wherein the attachment is configured to be coupled to the shaper at a selected angle.

72. The shaping system of claim 68, wherein the attachment is configured to be coupled to the shaper at a selected angle, and wherein the selected angle comprises an angle from the left ventricle to a heart valve.

73. The shaping system of claim 68, wherein the attachment comprises a selected length.

74. The shaping system of claim 68, wherein the attachment comprises a selected diameter.

75. The shaping system of claim 68, wherein the attachment is integrally included in the shaper.

76. The shaping system of claim 65, wherein the shaper has a substantially similar length as the appropriate left ventricle.

77. The shaping system of claim 65, wherein the shaper has a substantially similar width as the appropriate left ventricle.

78. The shaping system of claim 65, wherein the shaper has a substantially similar size as the appropriate left ventricle.

79. The shaping system of claim 65, wherein the shaper comprises an overall length substantially similar to an overall length of the appropriate left ventricle.

80. The shaping system of claim 65, wherein the shape of the shaper is used as a guide to allow a surgeon to reconstruct the left ventricle to the shape of the appropriate left ventricle during use.

81. A method for reconstructing an enlarged left ventricle, comprising:
opening the enlarged left ventricle;
placing a shaper into the enlarged left ventricle, the shaper having a shape substantially different than the shape of an appropriate left ventricle of a heart, wherein the shaper comprises an upper portion substantially similar in shape to an upper portion of the appropriate left ventricle, and wherein the shaper comprises an apex portion substantially similar in shape to an apex portion of the appropriate left ventricle;
pulling the enlarged left ventricle over the shaper;
suturing the left ventricle such that an interior surface of the left ventricle conforms to the shape of the appropriate left ventricle;
partially closing the opening;
removing the shaper from the left ventricle; and
completely closing the opening such that the enlarged left ventricle is reconstructed to the shape of the appropriate left ventricle.

82. The method of claim 81, further comprising suturing a patch to an interior surface of the left ventricle.

83. The method of claim 81, further comprising suturing a patch along at least one demarcation line.

84. The method of claim 81, further comprising excluding scar tissue from viable tissues.

85. The method of claim 81, wherein the shaper is used as a guide to reconstruct the left ventricle to the shape of the appropriate left ventricle.

86. The method of claim 81, wherein the shaper comprises an access port.

87. The method of claim 81, further comprising performing an endoscopy through an access port in the shaper.

88. The method of claim 81, further comprising accessing papillary muscles and/or chordae tendinae through an access port in the shaper.

89. The method of claim 81, further comprising measuring papillary muscles and/or chordae tendinae using graduated markings on the shaper.

90. The method of claim 81, further comprising correcting papillary muscles and/or chordae tendinae using graduated markings on the shaper.

91. The method of claim 81, further comprising aligning a heart valve with the left ventricle using an attachment coupled to the shaper.
